(19) United States
(12) Patent Application Publication
(10) Pub. No.: US 2013/0337445 A1
(43) Pub. Date: Dec. 19, 2013

(54) METHOD FOR THE CLINICAL DEVELOPMENT AND MEDICAL APPLICATION OF MULTIPLEX ASSAYS DERIVED FROM PATTERNS OF INDIVIDUAL BIOMARKERS OF OXIDATIVE OR NITROSATIVE STRESS

(71) Applicant: Norman A. Paradis, Putney, VT (US)

(72) Inventor: Norman A. Paradis, Putney, VT (US)

(21) Appl. No.: 13/921,613

(22) Filed: Jun. 19, 2013

Related U.S. Application Data
(60) Provisional application No. 61/661,418, filed on Jun. 19, 2012.

(51) Int. Cl. G01N 33/50

(52) U.S. Cl. CPC G01N 33/50
USPC 435/6.11; 436/71; 436/86; 435/7.92; 435/6.19; 435/15; 435/27; 435/25

(57) ABSTRACT

A method and apparatus for the discovery, development and clinical application of multiplex synthetic assays based on patterns of free radicals, indicators of oxidative or nitrosative stress, or indicators of the redox state of biologic fluids and tissue specimens. These individual measurements are combined into an optimized clinical biomarker using known well-known mathematical, machine learning techniques.

### REFERENCE TO PENDING PRIOR PATENT APPLICATION

This patent application claims benefit of pending prior U.S. Provisional Patent Application Serial No. 61/661,418, filed Jun. 9, 2012 by Norman A. Paradis for METHOD FOR THE CLINICAL DEVELOPMENT OF MULTIPLEX ASSAYS DERIVED FROM PATTERNS OF INDIVIDUAL BIOMARKERS OF OXIDATIVE OR NITROSATIVE STRESS (Attorney's Docket No. BARASH-4 PROV), which patent application is hereby incorporated herein by reference.

### FIELD OF THE INVENTION

This invention relates to medical assays in general, and more particularly to methods and apparatus for the discovery, development and clinical application of multiplex assays based on patterns of oxidative or nitrosative stress.

### BACKGROUND OF THE INVENTION

The maintenance of homeostasis through respiration, and the defense of the organism via innate and adaptive immunity, produces and utilizes pro-oxidant and pro-nitrosative reactive oxygen and nitrogen species. Such so-called “free radicals” have the capacity to accept or donate electrons, creating a tendency to react with other biomolecules. These molecules can act as important local messengers, allowing control of processes such as circulation, respiration, cell division and cell death. They are also important effector components of cell mediated immunity. However, free radicals can also react with other biologic molecules in a manner that is destructive of their function and harmful to the organism.

The concentration of reactive oxygen and nitrogen species within living tissues is a function of the balance between the production of pro-oxidant and pro-nitrosative molecules, environmental oxidative stress, environmental antioxidant nutrients, and the body's natural anti-oxidant defense mechanisms. Imbalances within these determinants may lead to abnormal patterns of free radicals, oxidative or nitrosative stress, and redox potential. Such imbalances are believed to be of central importance in health and disease, in particular in immunologically mediated, environmental and age related disease processes (see Berlett B S, Stadtman E R. Protein oxidation in aging, disease, and oxidative stress. J Biol Chem 1997; 272(33):20313-20316).

Oxidative damage may take the form of oxidation or peroxidation of biologic molecules. A peroxide being a compound containing an oxygen—oxygen single bond or the peroxide anion ([O—O]²⁻).

Uniplex measurement of free radicals, redox state, and oxidative or nitrosative stress in biologic specimens has been accomplished in a number of different ways:
1. Measurement of specific free radicals
2. Measurement of oxidative products
3. Measurement of peroxidation products
4. Measurement of oxidative or reductive potency
5. Measurement of “oxidizability” or oxidizing capacity
6. Concentration of specific OS promoters
7. Concentration of specific OS inhibiters (anti-oxidants)
8. Concentration of specific oxidized or peroxidized lipids
9. Concentration of oxidized or peroxidized proteins
10. Concentration of oxidized, peroxidized or damaged nucleic acids

Studies of the uniplex measurement of oxidative or nitrosative stress indicate that these processes are of central importance in many disease states. In particular diseases that include abnormalities of oxidative metabolism, immunity and cell death, such as visceral organ ischemia, heart failure, sepsis, and the various forms of shock. Accurate measurement of oxidative or nitrosative stress will likely be useful and important in medical diagnostics, and alteration of oxidative or nitrosative stress important in therapeutics.

The measurement of a patient's physical, chemical and anatomical properties is a central component of medical diagnosis. It has been appreciated by others that pathologic and non-pathologic alterations in the oxidation-reduction potential (redox) causes measurable changes in biologic macromolecules. Measurement of redox, markers of oxidative or nitro sative stress, and oxidative changes in biologic molecules has been undertaken in the study of almost all disease states and found to be important in many.

Because the abnormalities of oxidative or nitrosative stress in differing disease states derives from varying etiologies, it is assumed that the molecular and chemical patterns will be different in differing disease states. However, measurements of oxidative or nitrosative stress, and the assays which have been developed to date, have been limited to single parameters such as the redox potential, concentration of individual pro- or anti-oxidant molecules, or the concentration of specific free radicals, oxidized or peroxidized molecular species. Such individual uniplex indicators of oxidative or nitrosative stress may not have the sensitivity and specificity required for clinically useful medical assays.

Recently, there has been great interest in developing useful medical diagnostics based upon multiplex algorithms constructed from the measurement of multiple individual molecular species (see Kato K. Algorithm for in vitro diagnostic multivariate index assay. Breast Cancer 2009; 16(4):248-251). Such synthetic multiplex diagnostics hold the promise of medical tests for conditions in which no single molecule appears to diagnostically useful, and have been widely studied investigated in proteomics and genomics. These same approaches, however, have not been applied to the molecular changes associated with alterations in the redox and oxidative or nitrosative stress.

### SUMMARY OF THE INVENTION

A method and apparatus for the discovery, development and clinical application of multiplex synthetic assays based on patterns of free radicals, indicators of oxidative or nitrosative stress, or indicators of the redox state of biologic fluids and tissue specimens. These individual measurements are combined into an optimized clinical biomarker using known well-known mathematical, machine learning techniques.

In one preferred form of the present invention, there is provided a method for diagnosing, or determining the risk of, a medical condition, the method comprising:
measuring a plurality of biomarkers indicative of a patient's state of oxidative or nitrosative stress;
using a computational equation or algorithm to transform the measured plurality of oxidative or nitrosative stress biomarkers into a result which is indicative of the presence of, or the risk of, a medical condition in a specific individual.

In another preferred form of the present invention, there is provided a method for discovery, development or optimization of a multiplex biomedical assay, the method comprising:
measuring a plurality of biomarkers indicative of a patient's state of oxidative or nitrosative stress in the setting of clinically occurring or experimentally induced disease, disease mimics, or health, and using known mathematical or machine learning techniques to discover and optimize the computational algorithm or equation iteratively, using clinical or biomarker classifiers, such that the algorithm or equation has superior performance compared to any of the individual input measurements.

### DETAILED DESCRIPTION OF THE PRESENT INVENTION

This invention is a method and apparatus for the discovery, development and clinical application of multiplex synthetic assays based on patterns of free radicals, indicators of oxidative or nitrosative stress, or indicators of the redox state of biologic fluids and tissue specimens. These individual measurements are combined into an optimized clinical biomarker using known mathematical, machine learning techniques.

Components of the algorithm may include, but are not limited to categories such as:
1. Measurement of specific free radicals
2. Measurement of oxidative products
3. Measurement of peroxidation products
4. Measurement of oxidative or reductive potency
5. Measurement of oxidizability, or oxidizing capacity
6. Concentration of specific OS promoters
7. Concentration of specific OS inhibiters (anti-oxidants)
8. Concentration of specific oxidized or peroxidized lipids
9. Concentration of oxidized or peroxidized proteins
10. Concentration of oxidized, peroxidized or damaged nucleic acids
11. Concentration of nitrated proteins or nucleic acids.

Individual examples of uniplex assays from which the multiplex algorithm may be derived include, but are not limited to:
1. Assays for peroxidized lipids such as: Malondialdehyde (MDA), F2-isoprostanes, exhaled volatiles, CD, LOOH, TBARS;
2. Assays for damage or alteration of proteins such as protein carbonyls, 3-nitrotyrosine, GSH, Kidney Injury Molecule-1 KIM-1, Advanced Glycation End Products, Advanced Oxidation Protein Products (AOPP) Assay, BPDE Protein Adduct ELISA, Kidney Injury Molecule-1 (KIM-1) Assays, Oxidized/Nitrated Proteins, Protein Carbonyl Assays, Protein Nitration Assays and Reagents;
3. Assays for damage to nucleic acids such as 8-OH-Dg, 8-OXO-dG, 5-OH-mdU SCSA, Comet, and Tunnel, double stranded DNA breaks, 8-OHdG DNA Damage ELISA, 8-OHG RNA Damage ELISA, AP Sites Quantitation, BPDE DNA Adduct ELISA, Checkpoint Kinase Activity Assays, Comet Assay Kits and Slides, DNA Double-Strand Break Assay, Global DNA Methylation and Hydroxymethylation, UV Induced DNA Damage;
4. Assays for antioxidants, such as vitamin C, vitamin E, uric acid, carotenes, GPX, catalase, glutathione Catalase Activity Assay, Glutathione Assay, HORAC (Hydroxyl Radical Antioxidant Capacity) Assay, ORAC (Oxygen Radical Antioxidant Capacity) Assay, Superoxide Dismutase (SOD) Assay, Total Antioxidant Capacity (TAC) Assay;
5. Assays for oxidative potency, such as TRAP, FRAP, ORAC, TEAC;
6. Assays for oxidizability such as lag time, propagation, maximal rate, maximum CD;
7. Assays for nitrated proteins or nucleic acids; and
8. Assays as described above run on blood, serum, plasma, lymph, urine, cerebrospinal fluid, and exhaled breath, among others.

Any oxidative or nitrosative stress biomarker inputs to the mathematical model, or pattern of multiple inputs to the mathematical model, may be used in combination with any other input to create a synthetic biomarker whose diagnostic performance is superior to that of the individual inputs.

The invention itself is a pattern, or transformation, of individual biomarkers that achieves its medical utility through computational transformation of the measured parameters into a single synthetic clinically useful biomarker. The mathematical techniques used to create such models are well known to those familiar with the art. Particular examples would include linear, generalized, or survival regression modeling, decision tree modeling, random forest modeling, adaptive boosting modeling, support vector machines, neural networks, or a combination of these techniques, among others.

The biomarker pattern or algorithm is discovered based on the use of a clinical classifier, sometimes called a “gold standard”, that allows accurate identification of patients with and without the disease.

The specific computational tool utilized to develop the multiplex assay may not be of central and important to the development methodology. Commonly utilized software allows simultaneous comparison of many computational tools to identify the best performer based on the chosen clinical classifier.

Once classified patients, samples or data, are available, the multiplex pattern or algorithm is obtained through computational construction or transformation of the data into mathematical model, equation, or algorithm.

It is anticipated that physiologic measurements or patient characteristics may also be utilized in the final multiplex algorithm so as to improve the diagnostic performance. In a particular embodiment, the patient demographics or vital signs would be utilized as an input to optimize performance

Of particular importance to the discovery and optimization of the multiplex algorithm is that the computational tools are applied iteratively based on the classifier.

The oxidative or nitrosative stress biomarker algorithm may be adaptive, improving over time or as a function of feedback within an individual patient or epidemiologically useful unit. For example, the biomarker algorithm may be different in hospitals whose incidence of the disease of interest is different. Some inputs to the algorithm may be adaptable at the bedside based on changes in the patient's clinical state.

Temporal changes in the biomarker may be clinically useful. For instance, the change in one or more components over time or a comparison to baseline measurements may be is used in the algorithm.

The oxidative or nitrosative stress biomarker algorithm obtained may be used before and after administration of systemic or local physical or pharmacologic agents whose physiologic effects on the probability distribution is favorable to diagnostic performance.

Implementation

In one preferred form of the present invention, the method is implemented using a computational device, e.g., an appropriately programmed general purpose computer, a dedicated computer, etc., with the output of the computational device being displayed to the user.

Modifications Of The Preferred Embodiments

It should be understood that many additional changes in the details, materials, steps and arrangements of parts, which have been herein described and illustrated in order to explain the nature of the present invention, may be made by those skilled in the art while still remaining within the principles and scope of the invention.

What is claimed is:

1. A method for diagnosing, or determining the risk of, a medical condition, the method comprising:
measuring a plurality of biomarkers indicative of a patient's state of oxidative or nitrosative stress;
using a computational equation or algorithm to transform the measured plurality of oxidative or nitrosative stress biomarkers into a result which is indicative of the presence of, or the risk of, a medical condition in a specific individual.

2. A method for discovery, development or optimization of a multiplex biomedical assay, the method comprising:
measuring a plurality of biomarkers indicative of a patient's state of oxidative or nitrosative stress in the setting of clinically occurring or experimentally induced disease, disease mimics, or health, and using known mathematical or machine learning techniques to discover and optimize the computational algorithm or equation iteratively, using clinical or biomarker classifiers, such that the algorithm or equation has superior performance compared to any of the individual input measurements.

3. A method according to claim 1 wherein the result of computational algorithm or equation is transformed into a simpler index indicative of the presence of, or the risk of, a medical condition.

4. A method according to claim 1 wherein the temporal pattern of a plurality of oxidative or nitrosative stress biomarkers is an input to the computational process.

5. A method according to claim 1 in which the invention itself is a pattern, or transformation, of individual biomarkers that achieves its medical utility through mathematical construction or transformation of the measured parameters into a single synthetic clinically useful biomarker.

6. A method according to claim 1 wherein at least one of the individual assays measures free radicals.

7. A method according to claim 1 wherein at least one of the individual assays measures peroxidation products.

8. A method according to claim 1 wherein at least one of the individual assays measures one or more indicators of oxidative or nitrosative stress inhibiters (i.e. anti-oxidants).

9. A method according to claim 1 wherein at least one of the individual assays measures one or more indicators of oxidized, nitrated, peroxidized or damaged lipids, such as: Malondialdehyde (MDA), F2-isoprostanes, exhaled volatiles, CD, LOOH, TBARS.

10. A method according to claim 1 wherein at least one of the individual assays measures one or more indicators of oxidized, nitrated, peroxidized or damaged proteins, such as: protein carbonyls, 3-nitrotyrosine, GSH, Kidney Injury Molecule-1 KIM-1, Advanced Glycation End Products, Advanced Oxidation Protein Products (AOPP) Assay, BPDE Protein Adduct ELISA , Kidney Injury Molecule-1 (KIM-1) Assays, Oxidized/Nitrated Proteins, Protein Carbonyl Assays, Protein Nitration Assays and Reagents.

11. A method according to claim 1 wherein at least one of the individual assays measures one or more indicators of oxidized, nitrated, peroxidized or damaged nucleic acids, such as: 8-OH-Dg, 8-OXO-dG, 5-OH-mdU SCSA, Comet, and Tunnel, double stranded DNA breaks, 8-OHdG DNA Damage ELISA, 8-OHG RNA Damage ELISA, AP Sites Quantitation, BPDE DNA Adduct ELISA, Checkpoint Kinase Activity Assays, Comet Assay Kits and Slides, DNA Double-Strand Break Assay, Global DNA Methylation and Hydroxymethylation, UV Induced DNA Damage.

12. A method according to claim 1 wherein at least one of the individual assays measures antioxidants, such as: vitamin C, vitamin E, uric acid, carotenes, GPX, catalase, glutathione Catalase Activity Assay, Glutathione Assay, HORAC (Hydroxyl Radical Antioxidant Capacity) Assay, ORAC (Oxygen Radical Antioxidant Capacity) Assay, Superoxide Dismutase (SOD) Assay, Total Antioxidant Capacity (TAC) Assay.

13. A method according to claim 1 wherein at least one of the individual assays measures oxidative potency, such: as TRAP, FRAP, ORAC, TEAC.

14. A method according to claim 1 wherein at least one of the individual assays measures oxidizability, such as: lag time, propagation, maximal rate, maximum CD.

15. A method according to claim 1 wherein at least one of the individual assays measures fractional nitric oxide concentration in exhaled breath (FE_{NO}).

16. A method according to claim 1 wherein one or more of the input biomarkers is a physiologic measurements such as vital signs, or patient demographics, such as age, among others.

17. A method according to claim 1 wherein the computational algorithm or equation is adaptive, improving over time or as a function of feedback within an individual patient or epidemiologically useful unit.

18. A method according to claim 1 wherein the oxidative or nitrosative stress biomarkers are measured after administration of systemic, local, physical or pharmacologic or affinity agent or agents whose effects on the probability distribution is favorable to diagnostic performance.
